(19) United States
(12) Patent Application Publication
(10) Pub. No.: US 2009/0185976 A1
(43) Pub. Date: Jul. 23, 2009

(54) METHOD FOR INTRODUCING A CHEMICAL AGENT INTO THE SYSTEMIC CIRCULATION

(75) Inventor: Jackie R. See, Fullerton, CA (US)

Correspondence Address: CHRISTIE, PARKER & HALE, LLP, PO BOX 7068, PASADENA, CA, 91109-7068, US

(73) Assignee: Bio-Sphere Technology, Inc.

(21) Appl. No.: 12/191,215

(22) Filed: Aug. 13, 2008

Related U.S. Application Data
(63) Continuation of application No. 09/334,784, filed on Jun. 16, 1999, now abandoned.
(60) Provisional application No. 60/089,427, filed on Jun. 16, 1998.

(51) Int. Cl. A61K 49/00; A61K 9/127; A61K 38/28

(52) U.S. Cl. 424/9.1; 424/450; 514/3

(57) ABSTRACT

A method for introducing a drug, imaging agent and/or other chemical agent into the systemic circulation comprises providing a the drug, imaging agent and/or other chemical agent in liposomes having a mean diameter of less than about 200 nm, preferably less than about 80 nm, as determined by negative-staining transmission electron microscopy. The liposomes are orally administered. It has been found that liposomes of such small sizes are not absorbed by the macrophages in the Peyer's patches of the gut, but instead are introduced into the venous circulation from the Peyer's patches, and then through the portal vein or inferior vena cava system, through the heart, and into the systemic circulation.

### CROSS-REFERENCE TO RELATED APPLICATION(S)

The present application claims the benefit of U.S. patent application Ser. No. 09/334,784, filed Jun. 16, 1999, pending, which claims priority to provisional application U.S. provisional application 60/089,427, filed Jun. 16, 1998, all of which are hereby incorporated by reference. The application discloses and claims only subject matter disclosed in prior application and names an inventor or inventors named in the prior application. Accordingly, this application constitutes a continuation or division.

### BACKGROUND OF THE INVENTION

Currently, a wide variety of chemical agents, such as growth hormone, insulin, and imaging agents, which require introduction into the systemic circulation must be administered parenterally. However, under certain circumstances, parenteral administration is undesirable. Accordingly, a need exists for a method for introducing such agents into the systemic circulation by oral administration.

### SUMMARY OF THE INVENTION

The invention relates to methods for introducing a drug, imaging agent and/or other chemical agent into the systemic circulation. The drug, imaging agent and/or other chemical agent is introduced into liposomes having a mean diameter of less than about 200 nm, preferably less than about 100 nm, still more preferably less than about 80 nm, and even more preferably less than about 50 nm, as determined by negative-staining transmission electron microscopy. The liposomes are orally administered. It has been found that liposomes of such small sizes are not absorbed by the macrophages in the Peyer's patches of the gut, but instead are introduced into the venous circulation from the Peyer's patches, and then through the portal vein or inferior vena cava system, through the heart, and into the systemic circulation. In contrast, it has been discovered that larger liposomes are taken up by the macrophages in the Peyer's patches and broken down in the macrophages. The invention provides a method for orally introducing into the systemic circulation chemical agents, and in particularly chemical agents that heretofore could only be introduced into the systemic circulation parenterally.

### BRIEF DESCRIPTION OF THE DRAWINGS

FIG. 1 is an electron photomicrograph (magnification: 16,000) of liposomes in the cervical lymph tissue.

FIG. 2 is an electron photomicrograph (magnification: 16,000) of liposomes in the liver, which are partially lysed in comparison to the lyophilized liposomes visualized in saline before oral ingestion by the animals.

### DETAILED DESCRIPTION OF THE INVENTION

In one embodiment, the invention is directed to methods for introducing a drug, imaging agent and/or other chemical agent into the systemic circulation. The drug, imaging agent and/or other chemical agent is introduced into liposomes having a mean diameter of less than about 200 nm, preferably less than about 100 nm, still more preferably less than about 80 nm, and even more preferably less than about 50 nm, as determined by negative-staining transmission electron microscopy. The liposomes preferably have a mean diameter of at least about 20 nm. The liposomes are orally administered.

The most accurate method to measure the size of liposomes is direct observation by negative-staining transmission electron microscopy, particularly with liposomes less than 1 (one) micron in diameter. Individual liposomes can be viewed and the size and lamellarity (number of layers) can be viewed if they are lyophilized. The sizes can be determined as well as the range of sizes.

More specifically, negative staining is used to visualize viruses, cells, and large molecules, as well as other particles such as liposomes. The contrast in the transmission electron micrographs is provided by the osmium ion of the negative stain which scatters the electrons. Since liposomes are a series of phospholipid bilayers composed principally of carbon and hydrogen, relatively few electrons are scattered and the liposomes appear clearer than the negative contrast stain of the osmium ion. A suitable technique for negative-staining transmission electron microscopy is described in Haschmeyers, R. H. and Myers, R. S. Principles and Techniques of Electron Microscopy Biological Applications. (Hayat, M. A. editor, Van Nostrand Reichold Co., New York) Vol. 2, 1972, the entire disclosure of which is incorporated herein by reference.

Exemplary agents that can be incorporated into the liposomes include any chemical agent that alters a bodily function or acts as an imaging agent which outlines a bodily structure, such as agents for ultrasound, the electromagnetic spectrum, x-rays of any sort, magnetic resonance imaging, positron emission, or electron beam scanning. The invention is particularly useful for agents that heretofore were capable of only being administered parenterally for introduction into the systemic circulation, and more particularly for those agents that are effective at relatively low doses, e.g., in the microgram range. Particularly suitable chemical agents include, but are not limited to, apomorphine, growth hormone, insulin, vascular endothelial growth factors (VEGF), platelet activation factors (PAF), cytokines, endothelial adhesion factors, sulfonylurea agents (hypoglycemia inducing), macrolide antibiotics, protein fusion agents of animal or yeast origin, slow channel inhibitors, angiotension converting enzyme inhibitors, herbs, alpha or beta sympathetic stimulating or inhibition agents, corticosteriods, male and female hormonal agents, such as estrogen, testosterone, and aldosterone, granulocyte stimulating factors, megakaryocyte stimulating agents, eicosanoids, diuretics such as furosemide, MRI and CT imaging agents, plasmids containing DNA or RNA fragments, genes or portions thereof, biguanides, anticholinergic agents, dopaminergic agents, anticoagulants, heparin, immunomodulators, antineoplastic agents, toxoids, thrombolytic agents, antiarrhythmics, contraceptives, antilipemic agents, vasodilators, vasopressors, dopamine receptor agents, somatostatin analogues, serotonin receptor agonists, reuptake inhibitors, ergot derivatives, antihistamines, antivirals, immunosuppresives, histamine receptor antiagnostics, smooth muscle relaxants, oxytocics, carbonic anhydrate inhibitors, xanthine derivatives, leukotriene antagonists, lung surfactants, benzodiazepones, photo-sensitizing agents, sympatolytics, cytoprotective agents, antifungals, cerebral vasodilators, amebicides, analeptic agents, analgesics, aspirin, anxiolytic agents, salicylates, local anesthetics, angiotensin II receptor antagonists, anorectics, anthelminics, chemotherapeutic agents, antimalarials, antituberculosis agents, sulfonamides, leprostatics, anticonvulsants, antidepressants, anti-inflammatory agents, antiarthritics, antidiabetic agents, antihypertensive agents, biological response modifiers such as interferon, blood plasma fractions, antiplatelet agents, colony stimulating factors, iron, heparin antagonists, bone metabolism regulators, fertility agents, anti-inflammatory agents, gonadotropin releasing hormones and inhibitors, homeopathic preparations, mast cell stabilizers, nucleoside analogues, appetite suppressants, calcitonin, parasympatholytics and mimetics, antianxiety agents, radiopaque agents, steroidal anti-inflammatory agents, bronchodilators, antipsoriatic agents, smoking cessation aids, antispasmodics, weight control preparations, thyroid preparations, gadolinium and other paramagnetic magnetic resonance imaging agents, and gamma and beta radioisotope imaging agents.

The amount of chemical agent included in the liposomes depends on the particular chemical agent and the application for which it is to be used, as can be determined by one skilled in the art, and can range from 1 to 99 percent by weight of the liposomes.

The liposomes of the present invention may be made of any suitable phospholipid, glycolipid, derived lipid, and the like. Examples of suitable phospholipids include phosphatide choline, phosphatidyl serine, phosphatidic acid, phosphatidyl glycerin, phosphatidyl ethanolamine, phosphatidyl inositol, sphingomyelin, dicetyl phosphate, lysophosphatidyl choline and mixtures thereof, such as soybean phospholipids, and egg yolk phospholipids. Suitable glycolipids include cerebroside, sulphur-containing lipids, ganglioside and the like. Suitable derived lipids include choleic acid, deoxycholic acid, and the like. The presently preferred lipid for forming the liposomes is egg phosphatidylcholine.

The liposomes may be formed by any of the known methods for forming liposomes and may be loaded with a chemical agent according to known procedures. Known methods for forming liposomes containing chemical agents are described, for example, in U.S. Pat. No. 4,235,871 to Papahadjopoulos, et al., and Oral Microbiology and Immunology, 1994, 9:146-153, the disclosures of which are incorporated herein by reference.

Preparation of a Homogeneous Population May be Accomplished by Conventional techniques such as extrusion through a filter, the filter being either the straight path or tortuous path type. Other methods of treating liposomes to form a homogenous size distribution are ultrasonic exposure, the French press technique, hydrodynamic shearing, homogenization using, for example, a colloid mill or Gaulin homogenizer, and microfluidization techniques. Microfluidization is one presently preferred method. Other techniques involving sonication are also preferred.

Microfluidization is described, for example, in U.S. Pat. No. 4,533,254 to Cook, et al., which is incorporated herein by reference. In a preferred microfluidization procedure, the liposomal emulsion is forced at high pressure through a small diameter opening and splattered onto a wall and then collected. In sonication techniques, the raw materials for the liposomes, e.g., phospholipids, are combined with chemical agents, placed in a sonicator, and sonicated for a time, at a temperature and at a speed sufficient to obtain liposomes of the desired size.

In a preferred method, the liposomes, prior to administration, are treated to protect them against pH changes, micellization, lipases, and digestive enzymes as they pass through the stomach and enter the small intestine. Preferably the liposomes are lyophilized. Alternatively, the phospholipid (or any other constituent of lipid wall) can be treated with an additive, such as an enteric additive or a crosslinking agent, prior to formation of the liposome. The treated liposomes can then be packaged in a suitable form, such as a pill or capsule, for oral ingestion.

Lyophilization may be accomplished by any method known in the art. Such procedures are disclosed, for example, in U.S. Pat. No. 4,880,836 to Janoff, et al., the disclosure of which is incorporated herein by reference. Lyophilization procedures preferably include the addition of a drying protectant to the liposome suspension. The drying protectant stabilizes the liposome suspension. The drying protectant stabilizes the liposomes so that the size and content are maintained during the drying procedure and through rehydration. Preferred drying agents are saccharide sugars including dextrose, sucrose, maltose, manose, galactose, raffinose, trehalose lactose, and triose sugars which are preferably added in amounts of about 5% to about 20% and preferably about 10% by weight of the aqueous phase of the liposomal suspension. Dextrose, sucrose and maltose are presently preferred. Manitol may be used in conjunction with any of the saccharides. Additional preservatives such as BHT or EDTA, urea, albumin, dextran or polyvinyl alcohol may also be used.

Alternatively, or additionally, the liposomes containing the chemical agents may be packaged for oral administration in either a pill form or a capsule. An enteric coating is preferably applied to the liposomes containing the chemical agents to prevent breakdown in the stomach. The enteric coating may be made of any suitable composition. Suitable enteric coatings are described, for example, in U.S. Pat. Nos. 4,311,833 to Namikoshi, et al.; 4,377,568 to Chopra; 4,385,078 to Onda, et al.; 4,457,907 to Porter; 4,462,839 to McGinley, et al.; 4,518,433 to McGinley, et al.; 4,556,552 to Porter, et al.; 4,606,909 to Bechgaard, et al.; 4,615,885 to Nakagame, et al.; and 4,670,287 to Tsuji, all of which are incorporated herein by reference. Preferred enteric coating compositions include alkyl and hydroxyalkyl celluloses and their aliphatic esters, e.g., methylcellulose, ethylcellulose, hydroxyethylcellulose, hydroxypropylcellulose, hydroxybutylcellulose, hydroxyethylethylcellulose, hydroxyprophymethylcellulose, hydroxybutylmethylcellulose, hydroxypropylcellulose phthalate, hydroxypropylmethylcellulose phthalate and hydroxypropylmethylcellulose acetate succinate; carboxyalkylcelluloses and their salts, e.g., carboxymethylethylcellulose; cellulose acetate phthalate; polycarboxymethylene and its salts and derivatives; polyvinylalcohol and its esters, polycarboxymethylene copolymer with sodium formaldehyde carboxylate; acrylic polymers and copolymers, e.g., methacrylic acid-methyl methacrylic acid copolymer and methacrylic acid-methyl acrylate copolymer; edible oils such as peanut oil, palm oil, olive oil and hydrogenated vegetable oils; polyvinylpyrrolidone; polyethyleneglycol and its esters, e.g., and natural products such as shellac. Other preferred enteric coatings include polyvinylacetate esters, e.g., polyvinyl acetate phthalate; alkyleneglycolether esters of copolymers such as partial ethylene glycol monomethylether ester of ethylacrylate-maleic anhydride copolymer or diethyleneglycol monomethylether ester of methylacrylate-maleic anhydride copolymer, N-butylacrylate-maleic anhydride copolymer, isobutylacrylate-maleic anhydride copolymer or ethylacrylate-maleic anhydride copolymer; and polypeptides resistant to degradation in the gastric environment, e.g., polyarginine and polylysine. Mixtures of two or more of the above compounds may be used as desired.

The enteric coating material may be mixed with various excipients including plasticizers such as triethyl citrate, acetyl triethyl citrate, diethyl phthalate, dibutyl phthalate, dibutyl sebacate, dibutyl tartrate, dibutyl maleate, dibutyl succinate and diethyl succinate and inert fillers such as chalk or pigments. The composition and thickness of the enteric coating may be selected to dissolve immediately upon contact with the digestive juice of the intestine. Alternatively, the composition and thickness of the enteric coating may be selected to be a time-release coating which dissolves over a selected period of time, as is well known in the art.

Methods for preparing and coating lyophilized liposomes are described in U.S. patent application Ser. No. 08/920,374, entitled “Method for Inducing a Systemic Immune Response to an Antigen”, the entire disclosure of which is incorporated herein by reference.

In another embodiment, the invention is directed to a method for introducing a chemical agent into a channel created in an organ or the skin of a patient. Liposomes containing the chemical agent are administered to the patient. The liposomes have a mean diameter of less than about 200 nm, preferably less than about 100 nm, still more preferably less than about 80 nm, and even more preferably less than about 50 nm, as determined by negative-staining transmission electron microscopy. The liposomes preferably have a mean diameter of at least about 20 nm.

Methods for creating microscopic channels in organs or the skin are well known to those skilled in the art. For example, a laser dermal perforator and a technique for using the same are described in U.S. Pat. No. 5,908,416, the entire disclosure of which is incorporated herein by reference. A method for creating channels in the heart, for example, by transmyocardial laster revascularization is described by Frazier et al. in Texas Heart Institute Journal, Vol. 25. No. 1, pages 24-29 (1998), the entire disclosure of which is incorporated herein by reference. A similar technique is disclosed in U.S. Pat. No. 5,840,059, the entire disclosure of which is incorporated herein by reference. A technique for intra-operative myocardial revascularization is disclosed in U.S. Pat. No. 5,554,152, the entire disclosure of which is also incorporated herein by reference.

After the channels are created, the chemical agent is introduced into the channels in small liposomes, i.e., liposomes having a mean diameter of less than about 200 nm, preferably less than about 100 nm, still more preferably less than about 80 nm, and even more preferably less than about 50 nm, as determined by negative-staining transmission electron microscopy. In one embodiment, the liposomes are orally administered. In an alternative embodiment, the liposomes are administered by injection. When the liposomes are administered by injection, they can be administered, for example, in the arteries upstream from the organ of interest or in the venous system downstream from the organ of interest.

In a particularly preferred embodiment, an attractant is introduced into the channels as they are formed. The attractant is a second chemical agent that is capable of attracting the chemical agent that is to be introduced into the channels. For example, when the chemical agent to be introduced is an antibody, the attractant can be an antigen. When the chemical agent is a negatively charged molecule, the attractant can be a positively charged molecule capable of attracting the negatively charged molecule, and vice versa. The liposomes containing the chemical agent are then introduced into the systemic circulation and bloodstream of the patient by oral administration. When the liposomes reach the organ of interest, i.e., the organ having the channels containing the attractant, the attractant causes the liposomes to enter the channels.

### EXAMPLE

Four-week old male CD-1 mice (16-18 g) were obtained from Charles Rivers Farms, Wilmington, Mass.

Liposomes were prepared as follows: A solution of PyS DHPE was prepared in a test tube by dissolving 25 mg of PyS DHPE in 0.1 ml of chloroform. Lipid solution was then prepared in a separate test tube by combining 313 mg of DPPC, 72 mg of cholesterol, 14 mg of dicetylphosphate, 144 μL PyS DHPE solution, and 1.056 ml of chloroform, for a total volume of approximately 1.20 ml.

360 μl of lipid solution was aliquoted into three glass tubes. The solvent in each tube was evaporated to dryness with nitrogen gas. A maltose solution was prepared by dissolving 200 mg of maltose in 2.0 ml of water. To the maltose solution was added a chemical agent (either apomorphine or a ioxaglat, a contrast agent) or nothing (as a control). 300 μl of maltose solution (with or without the chemical agent) was added to the lipid tubes. Q.S. with water to 9.0 ml. The chemical agents were added so that the total amount of apomorphine in the lipid solution was about 1% by weight and the total amount of ioxaglat in the lipid solution was about 10% by weight.

To make liposomes having a mean diameter of approximately 50 nm, the solutions were passed through a microfluidizer up to twenty times. The solutions were aliquotted into ten vials, which were placed in a freezer at 10° C. overnight and subsequently lyophilized.

10 mice were orally fed approximately 1,000,000 liposomes in 10 nM sodium acetate (about 110 μg) by gavage tube. After 3 days, the mice were sacrificed, and the brains, neck lymph nodes, hearts, spleens, Peyer's patches, livers and small intestines harvested. The tissues were diced into sections less than about 1 mm by a straight-edge razor, fixed in 4% glutaraldehyde for 3 hours, and then washed four times in 2M phosphate buffer. The fixed samples were embedded in epoxy resin, cut by microtome, placed onto 400 mesh copper grids and examined under an electron microscope. Liposome size was verified by negative-staining electron microscopy. The liposomes were generally within the range of about 28 to about 95 nm, with approximately 95% of the liposomes within one standard deviation of 50 nm with 500 grids measured after the liposomes were lyophilized and the sizes measured.

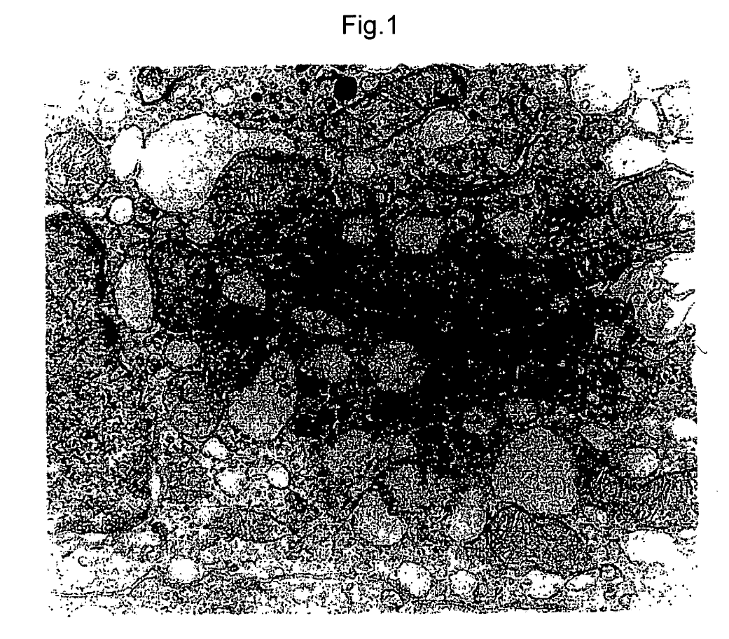

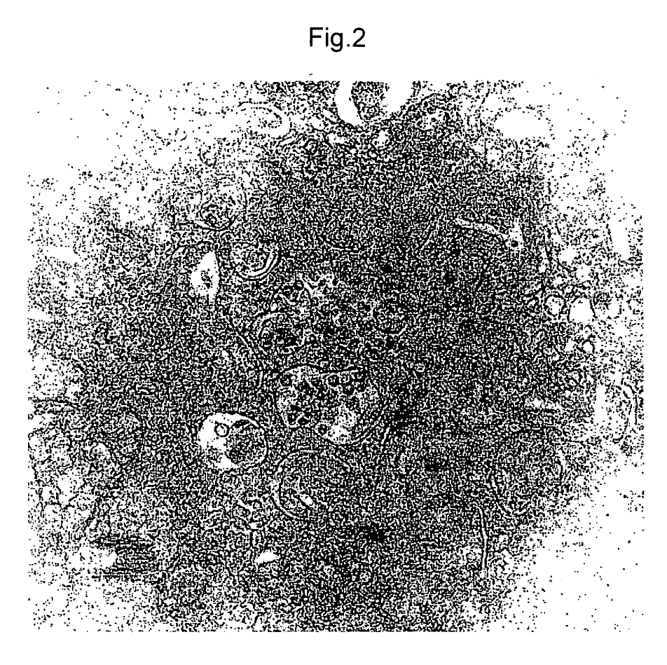

No liposomes were seen in the small bowel, Peyer's patches or spleen, suggesting that they were too small to be taken up by the mononuclear and polymorphonuclear cells and had passed through the venule system of the Peyer's patches into the systemic circulation. Liposomes were seen in the other tissue, although the slides of the brain tissue were somewhat unclear. The highest concentration of liposomes was in the liver. FIG. 1 shows 50 nm liposomes in cervical lymph tissue, and FIG. 2 shows 50 nm liposomes in the liver. The concentrations of liposomes observed were generally the same regardless of whether the liposomes contained apomorphine, contrast agent, or nothing, suggesting that the size of the liposome and not its contents determined the ability of the liposome to enter the systemic circulation.

The preceding description has been presented with reference to presently preferred embodiments of the invention. Workers skilled in the art and technology to which this invention pertains will appreciate that alterations and changes in the described structure may be practiced without meaningfully departing from the principal, spirit and scope of this invention. Accordingly, the foregoing description should not be read as pertaining only to the precise embodiments described, but rather should be read consistent with and as support to the following claims which are to have their fullest and fair scope.

What is claimed is:

1. A method for introducing a chemical agent into the systemic circulation of a patient comprising orally administering to the patient liposomes containing the chemical agent, wherein the liposomes have a mean diameter less than about 200 nm as measured by negative-staining electron microscopy.

2. A method according to claim 1, wherein the liposomes have a mean diameter less than about 100 nm as measured by negative-staining electron microscopy.

3. A method according to claim 1, wherein the liposomes have a mean diameter less than about 80 nm as measured by negative-staining electron microscopy.

4. A method according to claim 1, wherein the liposomes have a mean diameter less than about 50 nm as measured by negative-staining electron microscopy.

5. A method according to claim 1, wherein the chemical agent is an imaging agent.

6. A method according to claim 1, wherein the chemical agent is selected from the group consisting of apomorphine, growth hormone, insulin, vascular endothelial growth factors (VEGF), platelet activation factors (PAF), cytokines, endothelial adhesion factors, sulfonylurea agents (hypoglycemia inducing), macrolide antibiotics, protein fusion agents of animal or yeast origin, slow channel inhibitors, angiotension converting enzyme inhibitors, herbs, alpha or beta sympathetic stimulating or inhibition agents, corticosteriods, male and female hormonal agents, such as estrogen, testosterone, and aldosterone, granulocyte stimulating factors, megakaryocyte stimulating agents, eicosanoids, diuretics such as furosemide, MRI and CT imaging agents, plasmids containing DNA or RNA fragments, and genes and portions thereof.

7. A method according to claim 1, wherein the liposomes are lyophilized prior to administration.

8. A method according to claim 1, wherein the liposomes are contained within a pill or capsule.

9. A method according to claim 7, wherein the liposomes are contained within a pill or capsule.

10. A method for introducing a chemical agent into the systemic circulation of a patient comprising orally administering to the patient lyophilized liposomes containing the chemical agent, wherein the liposomes have a mean diameter less than about 80 nm as measured by negative-staining electron microscopy.

11. A method for introducing a chemical agent into the systemic circulation of a patient comprising orally administering to the patient liposomes containing the chemical agent, wherein the liposomes have a mean diameter less than about 200 nm as measured by negative-staining electron microscopy, whereby the liposomes are introduced into the patient's venous circulation from the patient's Peyer's patches.

12. A method according to claim 11, wherein the liposomes have a mean diameter less than about 100 nm as measured by negative-staining electron microscopy.

13. A method according to claim 11, wherein the liposomes have a mean diameter less than about 80 nm as measured by negative-staining electron microscopy.

14. A method according to claim 11, wherein the liposomes have a mean diameter less than about 50 nm as measured by negative-staining electron microscopy.

15. A method according to claim 11, wherein the liposomes are lyophilized prior to administration.

16. A method according to claim 11, wherein the liposomes are contained within a pill or capsule.

17. A method according to claim 15, wherein the liposomes are contained within a pill or capsule.

18. A method according to claim 1, further comprising, prior to administration of the liposomes, creating a channel in an organ or the skin of the patient, whereby the liposomes enter the channel.

19. A method according to claim 1, further comprising, prior to administration of the liposomes, creating a channel in the heart of the patient, whereby the liposomes enter the channel.

20. A method for introducing a chemical agent into a channel created in an organ or the skin of a patient, comprising orally administering to the patient liposomes containing the chemical agent, wherein the liposomes have a mean diameter of less than about 200 nm, whereby the liposomes are introduced into the systemic circulation and enter the channel in the organ or skin.

21. A method according to claim 20, wherein the liposomes have a mean diameter less than about 100 nm as measured by negative-staining electron microscopy.

22. A method according to claim 20, wherein the liposomes have a mean diameter less than about 80 nm as measured by negative-staining electron microscopy.

23. A method according to claim 20, wherein the liposomes have a mean diameter less than about 50 nm as measured by negative-staining electron microscopy.

24. A method according to claim 20, wherein the liposomes are lyophilized prior to administration.

25. A method according to claim 24, wherein the liposomes have a mean diameter less than about 80 nm as measured by negative-staining electron microscopy.
